(19) United States
(12) Patent Application Publication
(10) Pub. No.: US 2004/0209362 A1
(43) Pub. Date: Oct. 21, 2004

(54) Electrocompetent host cells

(75) Inventor: Alan L. Greener, San Diego, CA (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/804,969

(22) Filed: Mar. 19, 2004

Related U.S. Application Data
(60) Provisional application No. 60/455,804, filed on Mar. 19, 2003.

(51) Int. Cl. C12N 15/01; C12N 1/21

(52) U.S. Cl. 435/446; 435/252.33

(57) ABSTRACT

Provided are methods of generating electrocompetent bacterial cells, the methods involving the growth of the cells at hyperosmotic salt concentration. Also provided are methods of producing a transformed cell, methods of producing a recombinant polypeptide, and strains of E. coli that exhibit increased electrotransformation efficiency.

This application claims priority to the U.S. Provisional Application 60/455,804, filed Mar. 19, 2003, the entirety of which is incorporated herein by reference.

### FIELD OF THE INVENTION

The invention relates to host cells for the introduction of exogenous nucleic acid and methods of introducing exogenous nucleic acids to host cells, more particularly to host for the introduction of exogenous nucleic acids to bacteria by electroporation.

### BACKGROUND OF THE INVENTION

Introducing nucleic acids into E. coli and other host organisms is central to many types of experiments and analyses. There are several methods for introducing nucleic acids into various host cells, including, for example, incubating the host cells with co-precipitates of nucleic acids (Graham and van der Eb, Virology, 52: 456-467 (1973)), directly injecting nucleic acids into the nucleus of eukaryotic host cells (Diacumakos, Methods in Cell Biology, Vol. 7, eds. Prescott, D. M. (Academic Press) pp. 287-311 (1973), introducing nucleic acids via viral vectors (Hamer and Leder, Cell, 18: 1299-1302 (1979)), and using liposomes as a means of gene transfer (Fraley et al., J. Biol. Chem., 255: 10431-10435 (1980); Wong et al. Gene, 10: 87-94 (1980)). Introduction of nucleic acids into bacterial cells is performed by treating the cells with agents such as calcium chloride or rubidium chloride to induce the formation of pores in the cell wall and then incubating the treated cells with the nucleic acid.

Electroporation has also been used to transform host organisms, including E. coli. (Dower et al., Nucleic Acids Research, 16: 6127-6145 (1988); Taketo, Biochimica et Biophysica Acta, 949: 318-324 (1988); Chassy and Flickinger, FEMS Microbiology Letters, 44: 173-177 (1987); and Harlander, Streptococcal Genetics, eds. Ferretti and Curtiss (American Society of Microbiology, Washington, D.C.) pp. 229-233 (1987)).

An example of a typical electroporation method for bacterial cells is to grow bacteria in enriched media (of any sort) and to concentrate the bacteria by washing in 10% glycerol in water or non-conductive solution (Dower et al., 1988, U.S. Pat. No. 5,186,800). As discussed in U.S. Pat. No. 5,186,800, which is hereby incorporated by reference in its entirety, DNA is added to the cells and the cells are subjected to an electrical discharge, which temporarily disrupts the outer cell wall of the bacterial cells to allow DNA to enter the cells.

The electrical treatment to which the host cells are subjected during the process of electroporation typically results in the death of >90% of the host cells. However, it is believed that the majority of cells that survive electroporation take up the nucleic acids of interest.

In developing and refining electroporation methodology, researchers have identified factors that impact the efficiency of the transfer. These factors include, e.g., the electrical field strength, the pulse decay time, the pulse shape, the temperature in which the electroporation is conducted, the type of cell, the type of suspension buffer, and the concentration and size of the nucleic acid to be transferred (Andreason and Evans, Analytical Biochemistry, 180: 269-275 (1988); Sambrook, et al., Molecular Cloning: a Laboratory Manual, 2nd Edition, eds. Sambrook, et al. (Cold Spring Harbor Laboratory Press) pp. 1.75 and 16.54-16.55 (1989); Dower et al., (1988); Taketo (1988)).

### SUMMARY OF THE INVENTION

The invention provides improved methods of preparing electrocompetent cells, as well as improved strains of E. coli that better survive the electrotransformation process, and methods of making such strains.

The invention encompasses a method of generating electrocompetent cells, the method comprising: a) growing bacterial cells in culture medium at hyperosmotic salt concentration; and b) treating the cells to make them electrocompetent.

In one embodiment, the electrocompetent cells have an electrotransformation efficiency at least 30% greater than that for cells of the same bacterial strain grown under conditions of isoosmotic salt.

In another embodiment, the electrocompetent cells have an electrotransformation efficiency of at least 2×10¹⁰ cfu/μg DNA.

In another embodiment, the hyperosmotic salt concentration is 100 mM to 350 mM above isoosmotic. In another embodiment, the hyperosmotic salt concentration is 150 mM to 225 mM above isoosmotic. In another embodiment, the hyperosmotic salt concentration is 200 mM above isoosmotic.

In another embodiment, the step of growing bacterial cells at hyperosmotic salt concentration further comprises growing said cells under conditions of limited dissolved oxygen concentration. In a preferred embodiment, the conditions of limited dissolved oxygen concentration comprise a 1 to 10-fold reduction in dissolved oxygen relative to cultures grown under conditions of maximal aeration.

In another embodiment, step (b) comprises contacting the cells with glycerol. In a preferred embodiment, the cells are contacted with a 10% solution of glycerol in water. It is further preferred that the 10% solution of glycerol in water further comprises sorbitol.

In another embodiment, the method further comprises the step of drying the electrocompetent cells. It is preferred that upon re-hydration, the viable cells remain electrocompetent.

In another embodiment, step (a) comprises growing the bacterial cells to a final OD₅₅₀ of 0.75 to 0.85.

In another embodiment, the bacterial cells are Gram negative cells. In a preferred embodiment, the cells are E. coli.

In another embodiment, the culture medium comprises casein hydrolysate and/or maltose. In a preferred embodiment, the casein hydrolysate is present in the culture medium at a concentration of 11-15 g/liter. It is further preferred that the casein hydrolysate is present in the culture medium at a concentration of 11-12 g/liter, inclusive. It is further preferred that the maltose is present in the culture medium at a concentration of 0.1-0.3% (w/v). It is further preferred that the maltose is present in the culture medium at a concentration of 0.2-0.3% (w/v), inclusive.

The invention further encompasses a method of producing a transformed cell, the method comprising a) obtaining cells generated by growing bacterial cells in culture medium at hyperosmotic salt concentration, and treating the cells to make them electrocompetent; b) mixing the cells with a nucleic acid vector; subjecting the mixture of step (b) to an electrical treatment, and c) culturing the cells, such that a transformed cell is produced. In one embodiment, before making the bacterial cells electrocompetent, the cells are grown in bacterial medium at hyperosmotic salt concentration and under conditions of limited dissolved oxygen concentration.

The invention further encompasses a method of producing a recombinant polypeptide comprising: a) obtaining competent cells generated according to the method of claim 1; b) mixing the competent cells with a nucleic acid encoding the recombinant polypeptide; c) subjecting mixture of step (b) to an electrical treatment; and d) culturing the cells in a cell growth medium under conditions in which the cells produce the polypeptide.

In one embodiment, cells which have taken up the nucleic acid are separated from cells which have not taken up the nucleic acids.

In another embodiment, the recombinant polypeptide is isolated from the cells.

The invention further encompasses a biologically pure E. coli culture having all identifying characteristics of the E. coli strain 209K15 deposited with the American Type Culture Collection (ATCC) on Feb. 28, 2003 and assigned Accession No. PTA-5025 or mutants thereof that maintain increased transformation efficiency relative to the E. coli strain of ATCC Accesssion No. PTA-369.

In one embodiment, cells of the culture are electrocompetent.

In another embodiment, the culture comprises viable dried cells. In a preferred embodiment, the dried cells are electrocompetent upon re-hydration.

Definitions:

As used herein, the term “competent cell” refers to a cell which has the ability to take up and replicate an exogenous nucleic acid, and preferably to produce viable clonal progeny comprising the exogenous nucleic acid. An “electrocompetent cell” is a cell that has been treated such that it can take up exogenous nucleic acid when the cell is subjected to an electrical field. That is, bacterial cells are not inherently electrocompetent, but must be treated, for example by suspension in a non-conductive glycerol solution, to render them “electrocompetent” as the term is used herein.

As used herein, the phrase “hyperosmotic salt concentration” means a salt concentration outside a cell, e.g., in a culture medium, that is at least 10% higher than the salt concentration inside the cell.

As used herein, the phrase “conditions of limited dissolved oxygen concentration” means that the dissolved oxygen concentration of a culture or its medium is at least 5% less than the critical oxygen concentration for the organism being cultured. The “critical oxygen concentration” is that concentration of dissolved oxygen at which cells reach their maximal growth rate. “Maximal growth rate” means the rate of cell proliferation at which subsequent increases in dissolved oxygen content do not further increase growth rate. Culturing cells at a dissolved oxygen concentration below the critical oxygen concentration will therefore result in a growth rate below the maximal rate, also referred to as “oxygen-limited growth.” Where necessary, critical oxygen concentration for a given organism can be determined by one of skill in the art by culturing cells at varying dissolved oxygen concentrations and monitoring the growth rate of the cells. That concentration at which further increases in dissolved oxygen concentration do not increase the growth rate is the critical oxygen concentration. Methods of manipulating and measuring dissolved oxygen concentration are known in the art (see, e.g., John et al., 2003, Biotechnol. Bioeng. 81: 829-836) and described herein.

As used herein, the phrase “conditions of maximal aeration” refers to bacterial growth in which the concentration of dissolved oxygen is the highest possible with air, i.e., air saturation, for a given medium, at temperatures normally used for growth of that bacterium.

As used herein, the phrase “electrical treatment” means subjecting cells to an electric field. The term “electroporation” means subjecting cells to an electric field in the presence of exogenous nucleic acids such that the passage of exogenous nucleic acids occurs through pores in the cell membrane caused by the electric field.

The term “electrotransformation” means electroporating a population of cells in the presence of an exogenous nucleic acid which is taken up and replicated by at least a portion of those cells as a result of the electroporation.

As used herein, the term “transformation efficiency” refers to the number of transformed colonies formed with a given transformation reaction per unit mass of DNA added. Transformation efficiency is generally expressed in terms of transformed colonies per microgram of input DNA. Transformation efficiencies for embodiments of the electrocompetent cells described herein are preferably at least 1010 colonies/μg, and more preferably higher.

As used herein, the phrase “increased electrotransformation efficiency” refers to the property of a given cell strain to generate more transformed cells per unit of exogenous nucleic acid than a reference population of cells. The reference population can be a “parent strain” of cells, i.e., a cell strain as it existed before it was subjected to a selective pressure or otherwise genetically manipulated to derive a different strain having increased electrotransformation efficiency. Alternatively, the reference population can be a population of cells of the same genotype that have not been treated in a manner that effects increased transformation efficiency (e.g., the reference population was not subjected to altered salt or dissolved oxygen concentration during growth before electrotransformation). An “increase” as the term is used herein is at least 5%, but preferably at least 10%, 25%, 50%, 75%, 100% (i.e., two-fold), three fold, 10 fold, 50-fold, 100-fold or more relative to the electrotransformation efficiency of a reference cell strain.

As used herein, the phrase “electrical treatment-related cell death” refers to loss of cell viability associated with subjecting cells to an electric field, e.g., as in electroporation. While electroporation generally has higher transformation efficiency than chemically-mediated transformation, the process is very traumatic for the cells, often causing the death of 90% or more of the cells present. As used herein, the phrase “resistant to electrical treatment-related cell death” refers to the property of a given strain or mutant line of cells to experience a lower percentage of cell death when subjected to an electric field, e.g., as in electroporation, than a parent strain of cells. By “lower percentage” is meant at least 5% lower, but preferably at least 7%, 10%, 15%, 20%, 25%, 40%, 50%, 60%, 70%, 80%, 90% or more lower than the death observed in similarly treated cells of a parent strain.

As used herein, “selection” or “selecting” refers to a process of enriching for cells with desired heritable characteristics from a cell population, (e.g., such as resistance to electrical treatment-related cell death and/or enhanced transformation efficiencies) by identifying cells in a population with a desired characteristic.

As used herein, “clonal progeny” refers to genetically identical cells which result from the cell division of an isolated electrical treatment-resistant mutant cell.

As used herein, “mutation” refers to any alteration of the genetic constitution of a cell by changing the structure of the cell's hereditary material, deoxyribonucleic acid (DNA).

As defined herein, a “mutagen” is any agent or condition that can induce a mutation at frequencies greater than the spontaneous mutation rate for the organism.

As defined herein, “mutagenesis” refers to the process of generating a mutation. “Mutagenizing” also refers to the process of contacting a cell with a mutagen or otherwise treating a cell to generate a mutation (e.g., exposure to UV or ionizing radiation).

As defined herein, “mutants” or “mutant cells” are cells which comprise one or more mutations.

As used herein, the phrase “not proficient for recombination” or “recombination defective” means that a strain of cells lacks one or more functions instrumental in spontaneous recombination of nucleic acid within that strain. A non-limiting example of a recombination-defective cell is a RecA⁻E. coli cell.

As used herein, the phrase “recombination proficient” means that a cell strain can perform spontaneous recombination of nucleic acid within that strain.

As used herein, the phrase “limiting dilution” refers to a process for isolating clonal progeny of a single cell. Generally, the process involves diluting a population of cells such that a given volume (e.g., 10 μl) of the diluted cell suspension will in theory have less than one cell. When 10 μl aliquots of such a dilution are then used to inoculate separate culture containers, one aliquot per container, cells that grow in a given culture container can be considered clonal progeny of a single cell in the original inoculum. The process can be repeated to further ensure that the isolated cells are clonal.

As used herein, “% viability” means the number of cells that survive a given treatment, e.g., electroporation, divided by the number of cells subjected to the treatment, times 100. Survival is frequently determined by the ability to form colonies on an appropriate nutrient agar plate.

### DETAILED DESCRIPTION OF THE PREFERRED EMBODIMENTS

The invention provides methods of preparing cells for electrotransformation that have higher degrees of survival and higher transformation efficiencies relative to prior art methods. The invention also provides improved strains of E. coli that bear genetic changes that permit them to better survive the electrotransformation process, and methods of making such strains.

Preparation of Electrocompetent Cells:

I. Cells.

Cells to be prepared for and used in electrotransformation according to the methods described herein are bacterial cells. Preferred cells are Gram-negative bacterial cells. Gram-negative bacteria susceptible to electroporation by the methods described herein include, but are not limited to Enterobacteriaceae, such as Escherichia, Erwinia, Shigella, Salmonella, Proteus, and Yersinia; Brucellaceae, such as Brucella, Bordetella, Pasteurella, and Hemophilus; Azobacteraceae, such as Azotobacter; Rhizobiaceae, such as Rhizobium; Nitrobacteriaceae, such as Nitrosomonas. Nitrobacter, and Thiobacillus; Pseudomonadaceae, such as Pseudomonas and Acetobacter; Spirillaceae, such as Photobacterium, Zymonomas, Aermonas, Vibrio, Desulfovibrio, and Spirillum: and Actinomycetales, such as Mycobacterium, Actinomycees, Norcardia, and Streptomyces.

The present methods are applicable to bacteria which have previously been recognized as suitable hosts for receiving exogenous nucleic acids, particularly DNA plasmids. Such cloning hosts include E. coli, Pseudomonas species, including fluorocens and putida, and the like.

In a preferred embodiment, E. coli strains are rendered competent for electrotransformation by exogenous nucleic acids. Suitable E. coli strains include, but are not limited to, BB4, C600, DH5, DH5α, DH5α-E, DH5αMCR, DH5α5′IQ, DH5α5′, DH10, DH10B, DH10b/p3, DH10BAC, HB101, RR1, JV30, DH11S, DM1, LE392, SCS1, SCS110, Stab2, DH12S, MC1061, NM514, NM522, NM554, P2392, SURE®, SURE 2, XL1-Blue, XL1-Blue MRF, XL1-BlueMR, XL2-Blue, JM101, JM109, JM110/SCS110, NM522, TOPP strains, ABLE®, XLI-Red, BL21, TK B1, XL10-Gold® Cells, Restriction-Minus Competent Cells TK Cells, ABLE® strain, XlmutS strains, SCS110, AG1, TG1, SOLR™, XLOLR strain, Y1088, Y1089r, Y1090r-strains, WM100, and derivatives thereof. Information relating to the genotypes of these strains is available in the art and can be found, for example, on the World Wide Web at stratagene.com.

Bacterial strains can be obtained from numerous sources, including commercial sources and culture collections such as the American Type Culture Collection, Rockville, Md. 20852. Alternatively, they may be isolated from natural habitats. Once obtained, the bacterial cultures can be maintained in an active metabolic state or may be frozen or lyophilized until needed.

II. Growth of the Cells.

Improved tolerance of electrical treatment, with an accompanying improvement in transformation efficiency is achieved by growing bacterial cells under conditions of mild stress. In particular, when cells are grown in the presence of hyperosmotic salt concentrations in the medium, the cells will have increased resistance to the trauma caused by electrical treatment, e.g., electroporation. It has also been determined herein that the growth of cells under reduced dissolved oxygen concentration has an additional effect upon electrotransformation survival and electrotransformation efficiency.

Cells to be prepared for electrotransformation are grown in standard rich growth media suitable for the selected species of bacteria (e.g., LB, SOB, SOC, Psi broth, TB, TY, etc.), supplemented with a hyperosmotic concentration of salt (generally NaCl, although other salts, e.g., alkalai metal halides, alkaline earth metal halides, and ammonium salts and combinations thereof are contemplated). In order to have an effect, the hyperosmotic concentration of salt will be at least 10% greater than isoosmotic. Hyperosmotic concentrations of salt are preferably about 50 mM higher than isoosmotic, about 100 mM higher than isoosmotic, about 150 mM higher, about 175 mM higher, about 200 mM higher, about 225 mM higher, about 250 mM higher, about 275 mM higher, about 300 mM higher, about 325 mM higher or even about 350-400 mM higher, but are most preferably about 200 mM higher than isoosmotic. Determination of the most effective concentration of salt can be made by one of skill in the art without more than routine experimentation by, for example, growing a selected strain of bacteria in the presence of varying concentrations hyperosmotic salt within the ranges set forth herein above, and monitoring the electrotransformation efficiency of the cells.

Growth in the presence of a hyperosmotic salt concentration must be performed for a time sufficient to induce a mild stress response. Generally, cells to be used for electrotransformation are prepared by inoculating fresh medium at about a 1:100 dilution with a dense culture, e.g., an overnight culture, of the strain to be grown. This expansion culture is then incubated and monitored for growth by optical density (OD₅₅₀) measurement until approximately mid-log phase (OD₅₅₀≈0.75 to 0.85), at which time it is harvested and treated to render the cells competent. (The growth stage of the culture is known in the art to have an effect upon transformation efficiency. Refinements in the exact cell density at which cells are harvested can be made without undue experimentation by one of skill in the art for a given species or strain of bacteria.) While it is preferred that the duration of exposure to hyperosmotic salt be for the entire time in expansion culture, lesser times can be used. For example, one may increase the salt concentration after the expansion culture is initiated, rather than concurrent with initiation. In general, it is important that the cells be exposed to hyperosmotic salt for long enough to induce a mild stress response, and this will generally be at least the time required for one cell doubling. Optimal times for exposure to hyperosmotic salt can be adjusted by one of skill in the art with nothing more than routine experimentation, for example, by exposing the cells to hyperosmotic salt for varying amounts of time during the expansion culture before harvesting and comparison of the electrotransformation efficiencies resulting.

In addition to the effect of growth in the presence of hyperosmotic salt, it is discovered herein that culture under conditions of limited dissolved oxygen concentration has a positive effect on the survival and electrotransformation efficiency of bacterial cells. While not wishing to be held to any specific mechanism, it is thought that growth under such conditions also induces a stress response that causes the production of one or more protective agents by the cell.

For the embodiments disclosed herein in which dissolved oxygen concentration is limited, the dissolved oxygen concentration of a culture or its medium should be at least 5% less than the critical oxygen concentration for the bacterial organism being cultured. Reduction of dissolved oxygen concentration is preferably about 1-10-fold reduced relative to cultures grown under conditions of maximal aeration. Where necessary, critical oxygen concentration for a given organism can be determined by one of skill in the art by culturing cells at varying dissolved oxygen concentrations and monitoring the growth rate of the cells. That concentration at which further increases in dissolved oxygen concentration do not increase the growth rate is the critical oxygen concentration.

Methods of monitoring dissolved oxygen concentrations in solution are well known in the art. There are several general types of sensors. Electrochemical sensors use an electrochemical cell with a positive electrode (cathode) and a negative electrode (anode) connected by a salt bridge consisting of a saturated electrolyte solution. In this type of sensor, oxygen passes through a permeable membrane and is chemically reduced within the sensor cell, which reduction generates an electrical current that is detected. The current is proportional to the dissolved oxygen concentration.

Another type of dissolved oxygen sensor is the so-called “Clark” or “polarographic” sensor, which uses a gold or platinum cathode that is maintained at a polarizing voltage which causes the reduction of oxygen. The anode is generally silver. The amount of current from the anode to the cathode is directly related to the amount of dissolved oxygen present.

Finally, fiber optic sensors use an optical fiber with a sensor tip that has a bound layer of oxygen-sensitive fluorescent dye. Oxygen in a solution causes quenching of the fluorescence of the dye when the dye is stimulated with light transmitted down the optical fiber. The degree of quenching is proportional to the amount of oxygen in solution.

Dissolved oxygen meters of the various kinds are commercially available from, for example, Cole Parmer Instrument Co. (Vernon Hills, Ill.), Corning, Danfoss Analytical A/S (Sonderborg, Denmark), Extech Instruments Corp. (Waltham, Mass.), Fisher Scientific (Suwanee, Ga.), Hach Co. (Loveland, Colo.), Hanna Instruments (Woonsocket, R.I.), LaMotte Co. (Chestertown, Md.), Thermo Orion (Beverly, Mass.), Oxyguard International A/S (Birkerod, Denmark), Royce Instrument Corp. (New Orleans, La.), YSI Inc. (Yellow Springs, Ohio) and WTW Measurement Systems, Inc. (Ft. Myers, Fla.).

Bacterial cultures are generally grown in an incubator with rotary shaking. The rate of shaking directly influences the amount of dissolved oxygen introduced to the medium. Common wisdom has been that because the cells are subjected to harsh treatments in the process of rendering them competent and transforming them, the cells to be rendered competent should be as healthy as possible before treatment. Maximizing oxygen available to the cells tends to generate healthy cells that proliferate at maximal rates. Thus, cells are generally grown with vigorous shaking or stirring, in order to maximize the dissolved oxygen available to the cells. The inventors have discovered, contrary to this wisdom, that limitation of oxygen is beneficial for protection against electrical-treatment-related cell death. The preferred method of limiting dissolved oxygen is to reduce the speed of shaking during the expansion culture. For example, when a 250 ml Erlenmeyer flask is used as a culture vessel, shaking should be 225 rpm or less at a volume of 125 ml or more.

Another method of reducing or limiting dissolved oxygen concentration, which can be used in conjunction with or separately from reduction in shaking, is to increase the relative volume of the culture vessel. This has the effect of reducing the relative cross sectional surface area of the air/medium interface, and will limit the opportunity for air or oxygen to diffuse into the solution relative to standard culture conditions. For example, whereas a 250 ml Erlenmeyer flask might normally be used with an expansion culture volume of about 50 ml, increasing the volume to 125 ml or more will reduce the amount of surface area exposed relative to the volume of solution. It should also be appreciated that any change in the shape of the culture vessel that reduces the relative liquid/air interface surface area would have a similar effect—for example, changing a 250 ml culture from a 1 liter Erlenmeyer flask to a long cylindrical 1 liter vessel 7 cm in diameter will also reduce the cross sectional surface area of the air/medium interface and thereby reduce the dissolved oxygen concentration during incubation.

While manipulating the rate of shaking and/or the volume of culture relative to the vessel size are preferred methods of limiting dissolved oxygen concentration, more elaborate methods could also be employed if desired or necessary. For example, the incubator used could be a closed environment with a controlled atmosphere in which the amount of oxygen can be manipulated. In such an incubator, one would simply reduce the percentage of oxygen in the environment to below that normally present in air (about 21%). Cells could be grown with vigorous shaking if desired, but the presence of limiting oxygen in the incubator would limit the amount able to diffuse into the solution.

III. Making Cells Electrocompetent.

Bacterial cells are not inherently electrocompetent, but must be treated so as to render them electrocompetent. Cells can be rendered electrocompetent by any of a number of methods known in the art. Most methods involve suspending the cells in a glycerol/water solution to render them competent. The glycerol, which is non-polar, does not carry excess electrical charge into the cells, and glycerol is also a cryoprotectant that aids in preserving the cells if they are subsequently frozen for storage. U.S. Pat. No. 5,186,800, which is incorporated herein by reference, teaches rendering cells electrocompetent by centrifuging cells to pellet, and washing at least once, preferably more than once, in cold water or low conductivity solution (e.g., sucrose) to reduce salt concentration and electrical conductivity of the suspended cells. The washed cells are then resuspended in cold 10% glycerol at a concentration useful for electrotransformation (generally about 5×10⁹ to 1×10¹¹ cells/ml, preferably about 1×10¹⁰ to 5×10¹⁰ cells/ml) and aliquotted for storage or immediate use. The cells are then electrocompetent.

Another method is taught by U.S. Pat. No. 6,040,184, incorporated herein by reference. In this method, E. coli cells are grown in approximately 12 liters of medium in a 15 liter fermenter. The fermenter is cooled to 4° C. when cells reach the desired OD, and cells are concentrated by filtration. When the fluid level in the fermenter is reduced to 0.75 liters, buffer exchange is performed, running until 2 gallons of sterile 4° C. water has been exchanged. Another buffer exchange is then performed, running until 1 gallon of cold 15% glycerol has been exchanged. Cells treated in this manner are centrifuged gently (4000 rpm in a Sorvall swinging bucket rotor, 0° C., for 15 minutes) and resuspended in 35 ml of cold 15% glycerol. Electrocompetent ompetent cells made in this manner are then aliquotted for frozen storage (or immediate use). An improvement taught by the 6,040,184 patent is to add one or more sugars or sugar aldoses to the competent cells prior to electrotransformation. The sugars have a protective effect against electrical-treatment-related cell death.

As noted above, some cell freezing protocols call for the addition of glycerol to culture medium as a cryoprotective agent. However, the mere addition of glycerol to bacterial culture medium does not render the cells electrocompetent, partly because culture medium is not low conductivity and will therefore result in much higher cell death upon electroporation. To be effective, the glycerol treatment must be performed in the presence of a low conductivity solution. A “low conductivity solution” as the term is used herein has a conductivity of 30 μs/cm or lower. The conductivity of solutions used for electroporation is most often between about 12-19 μ/cm.

In one aspect, the invention provides a strain of E. coli that has genetic modifications permitting enhanced survival of electrical treatment. This strain was derived from ElectroTen Blue™ E. coli cells (Stratagene; ATCC Accession No. PTA-369) by mutagenesis and selection for enhanced survival of electroporation, coupled with manipulations to remove undesirable characteristics such as permissiveness for recombination. These cells exhibit enhanced survival and approximately two-fold higher electrotransformation efficiency relative to the parent ElectroTen Blue™ E. coli cell strain. Cells of this strain, designated 209K15, have been deposited with the American Type Culture Collection under the terms of the Budapest Treaty on Feb. 28, 2003 and have been assigned Accession No. PTA-5025.

2. Electroporation

Methods of electrotransformation or electroporation are well known in the art. For example, U.S. Pat. No. 5,186,800, and 6,338,965, incorporated herein by reference, describe electroporation methods. Briefly, bacterial cells, e.g., E. coli, are electroporated as follows. An aliquot (generally 20-40 μl) of electrocompetent cells at a concentration of 1×10¹⁰ to 5×10¹⁰ cells/ml is mixed with 1 μl of vector DNA suspended in 0.5×TE. The mixture is placed between the electrodes of a chilled electroporation cuvette. Electroporation can be performed using commercially available equipment, e.g., the GibcoBRL Cell-Porator™, or the Bio-Rad II GenePulser™. When a BioRad GenePulser II is used, electrical pulsing is performed, for example, at 1.7 kV, 200 Ω, and 25 mF. Following pulsing, the cuvette is immediately removed and SOC medium is added to 1 ml final volume. The suspension is then incubated in a 15 ml polypropylene tube and shaken at 250 rpm at 37° C. for 1 hour. 2.5 μl of the cell suspension is then spread onto an agar plate with a selective agent appropriate for the vector used, and the plate is incubated overnight at 37° C. Colonies are counted and transformation efficiency is determined. With a Cell-Porator™ electroporation system, the procedure is similar, only an initial power setting of 2.5 kV, 4000 Ω and 2 μF is used. Generally, manufacturers supply detailed instructions for the electroporation of bacteria using their equipment. One skilled in the art can readily determine conditions necessary for electroporation of bacteria using a given apparatus.

3. Determination of Electrotransformation Efficiency

Transformation efficiency can be determined by one skilled in the art. Briefly, in order to determine electrotransformation efficiency, the following is considered: The volume of cell suspension spread on the plate is divided by the total volume of cell suspension after the incubation in rich medium following electroporation, yielding the fraction of the suspension spread on the plate. The mass of DNA spread on the plate is the fraction of suspension spread times the mass of DNA (e.g., 2 ng) in the electrotransformation reaction. The number of colonies on the plate is then divided by the mass of DNA in the reaction to provide the electrotransformation efficiency, which is usually expressed in terms of colony forming units (cfu)/μg of DNA. If the cell suspension is diluted prior to plating (e.g., plated 10 μl of a 1:100 dilution after the 1 hr. incubation), the dilution factor is also considered in the calculation.

3. Storing Electrocompetent Cells

Electrocompetent cells can be stored either frozen or dried. Cells are stored frozen by snap freezing aliquots, e.g., 200 μl on a dry ice-ethanol bath. The glycerol in the solution used to render the cells electrocompetent also protects the cells during freezing. Frozen aliquots of cells are then stored at −80° C. until use.

Alternatively, electrocompetent cells can be dried, permitting storage at temperatures above −80° C. Methods of preparing dried bacterial cells are known in the art, e.g., WO 98/35018, incorporated herein by reference. A variety of other methods can be used, as described below. These methods include, but are not limited to, freeze-drying, air-drying, vacuum-drying, oven-drying, spray-drying, flash-drying, fluid bed-drying, and controlled atmosphere drying and are described, for example, in U.S. Pat. No. 5,728,574; U.S. Pat. No. 5,733,774; U.S. Pat. No. 5,200,399; U.S. Pat. No. 5,340,592; and U.S. Pat. No. 4,797,364, the entireties of which are incorporated by reference). In one embodiment, cells are dried at temperatures above freezing. In another embodiment, cells are dried at temperatures greater than or equal to 4° C. In still a further embodiment, cells are dried at room temperature (e.g., from 15-40° C.) under vacuum for 2-24 hours (e.g., 16 hours). In one embodiment, cells are dried under vacuum at non-atmospheric pressure, e.g., 1000-4000 mtorr.

In a preferred embodiment, cells are dried in the presence of a glass-forming matrix material. Suitable glass-forming matrix materials include carbohydrates, such as non-reducing sugars, which minimize oxidative damage to the cells. In one embodiment, the matrix material is a saccharide selected from the group consisting of trehalose, sucrose, melzitose, raffinose, alcohol derivatives thereof, and combinations thereof. In a preferred embodiment, the competent cells are contacted with a 20% carbohydrate solution, such as 20% trehalose, 20% sucrose, 20% melzitose, or 20% raffinose. In one embodiment, the cells are exposed to a solution which comprises 10% of two different carbohydrate solutions (e.g., 10% trehalose and 10% melzitose; 10% raffinose and 10% trehalose; 10% raffinose and 10% melzitose; 10% trehalose and 10% sucrose; 10% raffinose and 10% sucrose; or 10% melzitose and 10% sucrose).

In a preferred embodiment, a saccharide is used which does not crystallize upon drying and which comprises a Tg in the range of 10° C. to 80° C. In one embodiment, the glass-forming matrix material is a non-reducing carbohydrate selected from the group consisting of disaccharides, trisaccharides, oligosacharides and sugar alcohols thereof. Preferred saccharides include, but are not limited to, trehalose, raffinose, melezitose, sucrose, maltitol or combinations thereof. In one embodiment, a glass-forming saccharide is selected which hydrolyzes into a reducing sugar at a slow rate (e.g., such as trehalose). In another embodiment, a saccharide is selected which forms a hydrate when water is absorbed, thereby maintaining a high Tg (>15° C., and preferably greater than 40° C.) upon drying.

Other glass-forming matrix materials are known and are encompassed within the scope of the invention. These include, but are not limited to, dextran, polyethylene glyol, ficoll, and the like.

Dried electrocompetent cells are re-hydrated by the addition of water. Alternatively, cells can be re-hydrated in 4-5% trehalose solution or 10% glycerol. Cells are ready for electroporation immediately upon re-hydration.

4. Testing Cells for Electrocompetence:

Following the procedure to render cells electrocompetent, survival of electroporation and electrocompetence are tested as follows. 40 μl of chilled (on ice) electrocompetent cells are mixed with 1 μl of 10 μg/μl plasmid DNA (for testing, pUC 18 plasmid DNA is commonly used). The mixture is then transferred to a chilled electroporation cuvette with a 0.1 cm gap. The cuvette is placed into an electroporation apparatus and pulsed at the following settings: 1700 V, 17 kV/cm field strength, 600 Ω resistance and 25 μF capacitance. The mixture is immediately diluted with 960 μl of SOC medium, transferred to a 15 ml polypropylene tube (e.g., a Falcon™ 2059 tube) and incubated with shaking at 225-250 rpm for 1 hour at 37° C. Five to 100 ml of this 1 hr recovery culture is plated on an agar plate with the appropriate selective agent (e.g., ampicillin). Plates are incubated overnight, colonies are counted and electrotransformation efficiency is calculated as described herein. To determine the increase in survival and/or electrotransformation efficiency for a given set of growth and competence induction conditions relative to cells prepared under standard conditions, cells prepared under standard conditions (i.e., growth in isoosmotic salt with maximized (20-21%) oxygen saturation) are electroporated under the same conditions and electrotransformation efficiency is determined for comparison.

### EXAMPLES

### Example 1

### Growth of Cells in Hyperosmotic Salt and Under Conditions of Limited Dissolved Oxygen Permits Enhanced Electrotransformation Efficiency

E. coli strain ElectroTen Blue™ was grown under the following conditions. An overnight culture of ElectroTen Blue™ E. coli cells was inoculated into fresh SOB medium containing 200 mM NaCl (“high salt”) or 20 mM NaCl (“low salt,” or “standard” medium). For standard dissolved oxygen conditions, cells were cultured in a 50 ml volume in a 250 ml Erlenmeyer flask with shaking at 225-250 rpm. For conditions of limited dissolved oxygen, cells were cultured in a 125 ml volume in a 250 ml Erlenmeyer flask with shaking at 225-250 rpm. Cells were grown to an OD₅₅₀ of 0.5. Cells grown under both high salt/low O₂ and standard salt/standard O₂ were rendered electrocompetent by washing two times with cold water, followed by resuspension in 10% glycerol with 2.5% sorbitol. 40 μl of the electrocompetent cells were mixed with 1 μl of pUC18 (10 μg/μl), transferred to an electroporation cuvette (0.1 cm gap) and electroporated with a single pulse at 17 kV/cm field strength, 600 Ω resistance and 25 μF capacitance. The cells were diluted with 960 μl of SOC medium and transferred to a Falcon™ polypropylene tube and incubated for 1 hour with shaking at 225 rpm and 37° C. 10 μl of the cell suspension was plated on LB+Amp plates. The following results were obtained:

| | Electrotransformation |
|---|---|
| Cells and Conditions | Efficiency |
| ElectroTen Blue ™, standard salt and oxygen | 1.5 × 10¹⁰ cfu/μg |
| ElectroTen Blue ™, high salt, limited oxygen | 2.0 × 10¹⁰ cfu/μg |

### Example 2

### Cell Strains With Increased Resistance to Electrical Treatment

A mutant strain of ElectroTen Blue™ (Stratagene) E. coli cells has been identified by mutagenesis and selection for enhanced resistance to electrical treatment and subsequent manipulation to remove undesirable traits unrelated to resistance to electrical treatment. The mutant strain, termed 209K15, has been deposited with ATCC at Accession No. PTA-5025.

To examine the resistance to electrical treatment under standard and high salt/low dissolved oxygen conditions, overnight cultures of 209K15 E. coli cells or parental ElectroTen Blue™ E. coli cells were inoculated into separate cultures containing fresh SOB medium containing 200 mM NaCl (“high salt”) or 20 mM NaCl (“low salt,” or “standard” medium). For standard dissolved oxygen conditions, cells were cultured in a 50 ml volume in a 250 ml Erlenmeyer flask with shaking at 225-250 rpm. For conditions of limited dissolved oxygen, cells were cultured in a 125 ml volume in a 250 ml Erlenmeyer flask with shaking at 225-250 rpm. Cells were grown to an OD₅₅₀ of 0.5. Cells grown under both high salt/low O₂ and standard salt/standard O₂ were rendered electrocompetent by washing two times with cold water, followed by resuspension in 10% glycerol with 2.5% sorbitol. 40 μl of each preparation of electrocompetent cells were mixed with 1 μl of pUC18 (10 pg/μl), transferred to an electroporation cuvette (0.1 cm gap) and electroporated with a single pulse at 17 kV/cm field strength, 600 Ω resistance and 25 μF capacitance. The cells were diluted with 960 μl of SOC medium and transferred to a Falcon™ polypropylene tube and incubated for 1 hour with shaking at 225 rpm and 37° C. 10 μl of the cell suspension was plated on LB+Amp plates. The following results were obtained:

| | Electrotransformation |
|---|---|
| Cells and Conditions | Efficiency |
| ElectroTen Blue ™, standard salt and oxygen | 1.5 × 10¹⁰ cfu/μg |
| 209K, standard salt and oxygen | 3.0 × 10¹⁰ cfu/μg |
| ElectroTen Blue ™, high salt, limited oxygen | 2.0 × 10¹⁰ cfu/μg |
| 209K, high salt, limited oxygen | 5.0 × 10¹⁰ cfu/μg |

### Other Embodiments

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of generating electrocompetent cells, said method comprising:
a) growing bacterial cells in culture medium at hyperosmotic salt concentration; and
b) treating said cells to make them electrocompetent.

2. The method of claim 1 wherein said electrocompetent cells have an electrotransformation efficiency at least 30% greater than that for cells of the same bacterial strain grown under conditions of isoosmotic salt.

3. The method of claim 1 wherein said electrocompetent cells have an electrotransformation efficiency of at least 2×10¹⁰ cfu/μg DNA.

4. The method of claim 1 wherein said bacterial cells are Gram negative cells.

5. The method of claim 1 wherein said bacterial cells are E. coli cells.

6. The method of claim 1 wherein said hyperosmotic salt concentration is 100 mM to 350 mM above isoosmotic.

7. The method of claim 1 wherein said hyperosmotic salt concentration is 150 mM to 225 mM above isoosmotic.

8. The method of claim 1 wherein said hyperosmotic salt concentration is 200 mM above isoosmotic.

9. The method of claim 1, wherein said step of growing bacterial cells at hyperosmotic salt concentration further comprises growing said cells under conditions of limited dissolved oxygen concentration.

10. The method of claim 9 wherein said conditions of limited dissolved oxygen concentration comprise a 1 to 10-fold reduction in dissolved oxygen relative to cultures grown under conditions of maximal aeration.

11. The method of claim 1 wherein step (b) comprises contacting said cells with glycerol.

12. The method of claim 1 wherein said cells are contacted with a 10% solution of glycerol in water.

13. The method of claim 12 wherein said 10% solution of glycerol in water further comprises sorbitol.

14. The method of claim 1 further comprising the step of drying said electrocompetent cells.

15. The method of claim 14 wherein upon re-hydration, the viable cells remain electrocompetent.

16. The method of claim 1 wherein step (a) comprises growing said bacterial cells to a final OD₅₅₀ of 0.45 to 0.5.

17. The method of claim 1 wherein said culture medium comprises casein hydrolysate and/or maltose.

18. The method of claim 17 wherein said casein hydrolysate is present in said culture medium at a concentration of 11-15 g/liter.

19. The method of claim 17 wherein said casein hydrolysate is present in said culture medium at a concentration of 11-12 g/liter, inclusive.

20. The method of claim 17 wherein said maltose is present in said culture medium at a concentration of 0.1-0.3% (w/v).

21. The method of claim 17 wherein said maltose is present in said culture medium at a concentration of 0.2-0.3% (w/v), inclusive.

22. A method of producing a transformed cell, said method comprising
a) obtaining electrocompetent cells generated according to the method of claim 1;
b) mixing said electrocompetent cells with a nucleic acid encoding said recombinant polypeptide;
c) subjecting the mixture of step (b) to an electrical treatment; and
d) culturing said cells, such that a transformed cell is produced.

23. A method of producing a recombinant polypeptide comprising:
a) obtaining electrocompetent cells generated according to the method of claim 1;
b) mixing said electrocompetent cells with a nucleic acid encoding said recombinant polypeptide;
c) subjecting the mixture of step (b) to an electrical treatment; and
d) culturing said cells in a cell growth medium under conditions in which the cells produce said polypeptide.

24. The method of claim 23, in which cells which have taken up said nucleic acid are separated from cells which have not taken up said nucleic acids.

25. The method of claim 23, wherein said recombinant polypeptide is isolated from said cells.

26. A biologically pure E. coli culture having all identifying characteristics of the E. coli strain 209K15 deposited with the American Type Culture Collection (ATCC) and assigned Accession No. PTA-5025, or mutants thereof that maintain increased transformation efficiency relative to the E. coli strain of ATCC Accession No. PTA-369.

27. An electrocompetent cell according to claim 26.

28. A viable dried cell according to claim 26.

29. The cell of claim 27 that is electrocompetent upon re-hydration.
